(19) United States
(12) Patent Application Publication
(10) Pub. No.: US 2023/0165630 A1
(43) Pub. Date: Jun. 1, 2023

(54) SYSTEM AND METHOD FOR TISSUE PUNCTURE

(71) Applicant: Boston Scientific Medical Device Limited, Galway (IE)

(72) Inventors: Lauren Koon, Mississauga (CA); Jackie Leung, Richmond Hill (CA)

(21) Appl. No.: 18/072,328

(22) Filed: Nov. 30, 2022

Related U.S. Application Data
(60) Provisional application No. 63/284,302, filed on Nov. 30, 2021.

(51) Int. Cl. A61B 18/14; A61B 18/12; A61B 18/16; A61M 29/02

(52) U.S. Cl. CPC A61B 18/1477; A61B 18/16; A61B 18/1233; A61M 29/02; A61B 2018/00839; A61B 2018/1475

(57) ABSTRACT

A system for tissue puncture includes a radiofrequency (RF) generator, an RF puncture device, and at least a first intracorporeal grounding (IG) electrode. RF generator includes an RF output port and a ground return port. The RF puncture device includes an elongate member having a shaft and a tip. The tip includes an intracorporeal RF puncture electrode that is positionable adjacent a target site within a patient’s body, and the shaft includes a first electrical conductor that is electrically connected to the intracorporeal RF puncture electrode and is electrically connectable to the RF output port for delivering RF energy from the RF generator to the intracorporeal RF electrode. The IG electrode is positionable within the patient’s body proximate the target site, and is electrically connectable to the ground return port for returning current to the RF generator.

### CROSS REFERENCE TO RELATED APPLICATION

This application claims priority to U.S. Provisional Pat. Application Serial No. 63/284,302, filed Nov. 30, 2021, which is herein incorporated by reference in its entirety.

### FIELD

This document relates to medical procedures involving tissue puncture. More specifically, this document relates to systems and methods for tissue puncture using radiofrequency energy.

### SUMMARY

The following summary is intended to introduce the reader to various aspects of the detailed description, but not to define or delimit any invention.

Systems for tissue puncture are disclosed. According to some aspects, a system for tissue puncture includes a radiofrequency (RF) generator having an RF output port and a ground return port. The system further includes an RF puncture device including an elongate member having a shaft and a tip. The tip includes an intracorporeal RF puncture electrode that is positionable adjacent a target site within a patient’s body, and the shaft includes a first electrical conductor that is electrically connected to the intracorporeal RF puncture electrode and is electrically connectable to the RF output port for delivering RF energy from the RF generator to the intracorporeal RF electrode. The system further includes at least a first intracorporeal grounding electrode that is positionable within the patient’s body proximate the target site. The first intracorporeal grounding electrode is electrically connectable to the ground return port for returning current to the RF generator.

In some examples, the system further includes an intracorporeal accessory that includes the first intracorporeal grounding electrode.

In some examples, the intracorporeal accessory includes a sheath though which the RF puncture device is advanceable to position the RF puncture electrode at the target site. The sheath can have a sheath distal portion that is positionable proximate the target site and that defines a sheath distal end, a sheath proximal portion that is opposite the sheath distal portion and that defines a sheath proximal end, a sheath sidewall extending between the sheath distal end and the sheath proximal end, and a sheath lumen defined by the sheath sidewall and extending between the sheath distal end and the sheath proximal end. The first intracorporeal grounding electrode can be fixed to the sheath sidewall in the sheath distal portion, and the sheath can further include a ground return wire for electrically connecting the first intracorporeal grounding electrode to the ground return port. The first intracorporeal grounding electrode can include a first ring electrode that extends circumferentially around the sheath sidewall.

In some examples, the intracorporeal accessory includes a dilator through which the RF puncture device is advanceable to position the RF puncture electrode at the target site. The dilator can include a dilator distal portion that tapers in diameter towards a dilator distal end, a dilator proximal portion that is opposite the dilator distal portion and that defines a dilator proximal end, a dilator sidewall extending between the dilator distal end and the dilator proximal end, and a dilator lumen defined by the dilator sidewall and extending between the dilator distal end and the dilator proximal end. The first intracorporeal grounding electrode can fixed to the dilator sidewall in the dilator distal portion, and the dilator can further include a ground return wire for electrically connecting the first intracorporeal grounding electrode to the ground return port. The first intracorporeal grounding electrode can include a first ring electrode that extends circumferentially around the dilator sidewall.

In some examples, the intracorporeal accessory includes a diagnostic catheter having a catheter distal portion that is positionable proximate the target site and that defines a catheter distal end, a catheter proximal portion that is opposite the catheter distal portion and that defines a catheter proximal end, and a catheter sidewall extending between the catheter distal end and the catheter proximal end. The first intracorporeal grounding electrode can be fixed to the catheter sidewall in the catheter distal portion, and the catheter can further include a ground return wire for electrically connecting the first intracorporeal grounding electrode to the ground return port.

In some examples, the RF puncture device includes the first intracorporeal grounding electrode. The shaft can include a ground return wire that is electrically connected to the intracorporeal grounding electrode and that is electrically connectable to the ground return port for returning the current to the RF generator. The first intracorporeal grounding electrode can include a first ring electrode that is received on the shaft.

In some examples, the system can further include a dilator though which the RF puncture device is advanceable to position the RF puncture electrode at the target site. The dilator can include a dilator distal portion that tapers in diameter towards a dilator distal end, a dilator proximal portion that is opposite the dilator distal portion and that defines a dilator proximal end, a dilator sidewall extending between the dilator distal end and the dilator proximal end, and a dilator lumen defined by the dilator sidewall and extending between the dilator distal end and the dilator proximal end. In the dilator distal portion, the dilator sidewall can include a first window extending radially therethrough from an outer surface of the dilator to the dilator lumen. When the RF puncture device is advanced through the dilator to position the RF puncture electrode at the target site, the first intracorporeal grounding electrode can be aligned with the first window.

In some examples, the RF puncture device further includes a second intracorporeal grounding electrode. The system can further include a sheath having a sheath distal portion that is positionable proximate the target site and that defines a sheath distal end, a sheath proximal portion that is opposite the sheath distal portion and that defines a sheath proximal end, a sheath sidewall extending between the sheath distal end and the sheath proximal end, and a sheath lumen defined by the sheath sidewall and extending between the sheath distal end and the sheath proximal end. The RF puncture device and the dilator can advanceable through the sheath lumen to position the dilator distal portion proud of the sheath distal end and to position the RF puncture electrode proud of the dilator distal end and the sheath distal end and at the target site. In the sheath distal portion, the sheath sidewall can include a second window extending radially therethrough from an outer surface of the sheath to the sheath lumen. When the dilator is advanced through the sheath and the RF puncture device is advanced through the dilator to position the RF puncture electrode at the target site, the second intracorporeal grounding electrode can be aligned with the second window.

In some examples, the first intracorporeal grounding electrode has a larger surface area than the RF puncture electrode.

In some examples, the system further includes an electroanatomical mapping (EAM) system to which the first intracorporeal grounding electrode is electrically connectable for use of the first intracorporeal grounding electrode as an EAM electrode.

In some examples, the system further includes a switching device. The RF puncture electrode can be electrically connectable to the RF output port via the switching device. The first intracorporeal grounding electrode can be electrically connectable to the EAM system via the switching device, and electrically connectable to the ground return port via the switching device. The switching device can be configured to allow the first intracorporeal grounding electrode to be electrically connected to only one of the ground return port and the EAM system at a given time.

Methods for tissue puncture are also disclosed. According to some aspects, a method for tissue puncture includes: (a) advancing a radiofrequency (RF) puncture device towards a target site within a patient’s body and positioning an intracorporeal RF puncture electrode of the RF puncture device in contact with the target site; (b) advancing a first intracorporeal grounding electrode into the patient’s body and positioning the first intracorporeal grounding electrode proximate and spaced from the target site; (c) delivering RF energy from an RF outlet port of an RF generator to the RF puncture electrode, to puncture the target site; and (d) returning current to the first intracorporeal grounding electrode and delivering the current from the first intracorporeal grounding electrode to a ground return port of the RF generator.

In some examples, in step (a), the intracorporeal RF puncture electrode is positioned on a first side of the target site, and in step (b), the first intracorporeal grounding electrode is positioned on the first side of the target site.

In some examples, in step (a), the intracorporeal RF puncture electrode is positioned in a body cavity, and in step (b), the first intracorporeal grounding electrode is positioned in the body cavity. The body cavity can be the right atrium.

In some examples, step (b) includes advancing a sheath into the patient’s body. A distal portion of the sheath can include the first intracorporeal grounding electrode. Step (a) can include advancing the RF puncture device through the sheath.

In some examples, step (b) includes advancing a dilator into the patient’s body. A distal portion of the dilator can include the first intracorporeal grounding electrode. Step (a) can include advancing the RF puncture device through the dilator.

In some examples, step (b) includes advancing a diagnostic catheter into the patient’s body. A distal portion of the diagnostic catheter can include the first intracorporeal grounding electrode.

In some examples, a distal portion of the RF puncture device includes the first intracorporeal grounding electrode, and step (a) and step (b) are carried out concurrently by advancing the RF puncture device.

In some examples, the method further includes, before or after steps (c) and (d), connecting the first intracorporeal grounding electrode to an electroanatomical mapping system and using the first intracorporeal grounding electrode for electroanatomical mapping.

In some examples, the method further includes, before or after steps (c) and (d), connecting the RF puncture electrode to an electroanatomical mapping system and using the RF puncture electrode for electroanatomical mapping.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example system for tissue puncture including an RF generator, an RF puncture device, a dilator, and a sheath;

FIG. 2 is a perspective view of the RF puncture device of the system of FIG. 1;

FIG. 3 is a cross-section taken along line 3-3 in FIG. 2;

FIG. 4 is a cross-section taken through the distal portion of the sheath of the system of FIG. 1;

FIG. 5 is a schematic view showing the system of FIG. 1 in use;

FIG. 6 is a cross-section taken through the distal portion of another example dilator;

FIG. 7 is a schematic view showing the dilator of FIG. 6 is use in a system for tissue puncture;

FIG. 8 is a perspective view of another example system for tissue puncture in use;

FIG. 9 is a cross-section taken through the distal portion of another example RF puncture device;

FIG. 10 is a schematic view showing the RF puncture device of FIG. 9 in use in a system for tissue puncture;

FIG. 11 is a schematic view showing the RF puncture device of FIG. 9 in use in another system for tissue puncture; and

FIG. 12 is a perspective view of an example system for tissue puncture including an RF generator, an RF puncture device, a dilator, a sheath, an electroanatomical (EAM) mapping system, and a switching device.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

As used herein, the term “intracorporeal” refers to a procedure that occurs within a human body, or a device (or element thereof) that is used or is intended for use within the human body. Atrial perforation with the systems and apparatuses described herein is an example of an intracorporeal procedure. The puncture electrodes and grounding electrodes described herein are examples of intracorporeal elements.

Generally disclosed herein are apparatuses, systems, methods for tissue puncture. The methods generally involve positioning a radiofrequency (RF) puncture electrode of an RF puncture device against a target tissue (e.g. an atrial septum), and delivering energy from an RF generator to the RF puncture electrode to puncture the target tissue. Such procedures can be carried out, for example, as a medical treatment, or to gain access to the left atrium for a subsequent medical treatment. In the methods disclosed herein, an intracorporeal grounding (IG) electrode is used for returning current to the RF generator. The IG electrode can be positioned in the patient’s body, proximate the RF puncture electrode but spaced from both the RF puncture electrode and the target tissue. For example, the IG electrode can be positioned in the right atrium, proximally of the RF puncture electrode. As described in further detail below, this can be achieved, for example, by incorporating the IG electrode into the RF puncture device itself, or into an intracorporeal accessory such as sheath through which the RF puncture device is advanced, a dilator through which the RF puncture device is advanced, or a diagnostic catheter used concurrently in the medical procedure.

Providing an IG electrode that is positionable proximate the RF puncture electrode can enhance safety, as electrical energy need not travel a large distance through the body in order to complete the electrical circuit. Thus, the risks associated with leakage currents (e.g. physiological response, nerve stimulation, burns, and interference with other electronics) are reduced. Furthermore, by providing an IG electrode that is positionable proximate the RF puncture electrode, it is believed that a relatively low amount of power/current can be used for tissue puncture. This in turn can allow for the RF puncture electrode to be relatively small and for the RF puncture device to have a relatively thin layer of insulation, which can result in an RF puncture device of relatively small diameter. A small diameter RF puncture device may be relatively atraumatic, and may have additional uses (e.g. it may have an additional use as a diagnostic wire in coronary vessels). In this embodiment, the direction of the current moves backwards. In other words, the direction of current moves from the RF puncture electrode to the proximal IG electrodes.

In an alternative embodiment, the assembly may be constructed to have the proximal electrode(s) as delivering RF energy while the distal-most electrode may be configured as a ground. In this embodiment, the direction of the current is reversed as it moves from the proximal RF electrode(s) to the distal ground electrode. In other words, the direction of the current moves in a forward direction (proximal to distal). In some situations, this reversal of current direction may be more efficacious for puncturing tissue as the current density field is now applied towards the tissue rather than away from the tissue.

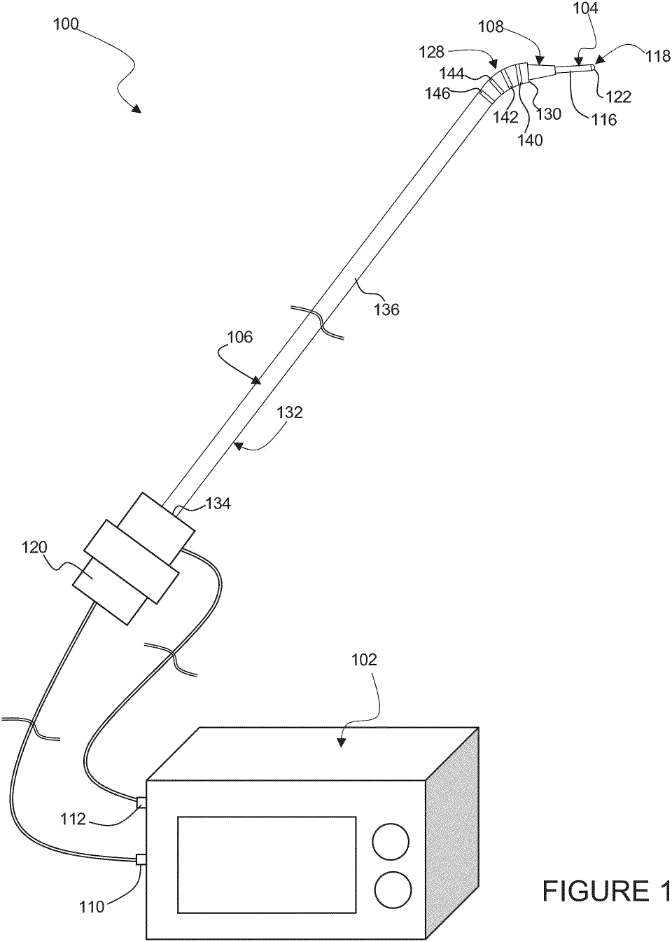

Referring now to FIG. 1, an example system 100 for tissue puncture is shown. As shown, the system 100 includes an RF generator 102, an RF puncture device 104, a sheath 106, and a dilator 108.

Referring still to FIG. 1, in the example shown, the RF generator 102 includes an RF output port 110 to which the RF puncture device 104 is electrically connectable, and a ground return port 112 to which a grounding electrode (described in further detail below) is electrically connectable. RF generators are commercially available, and are not described in detail herein. One such example is available from Baylis Medical Company, Inc. (Montreal, Canada) under the name “RFP-100A RF Puncture Generator”.

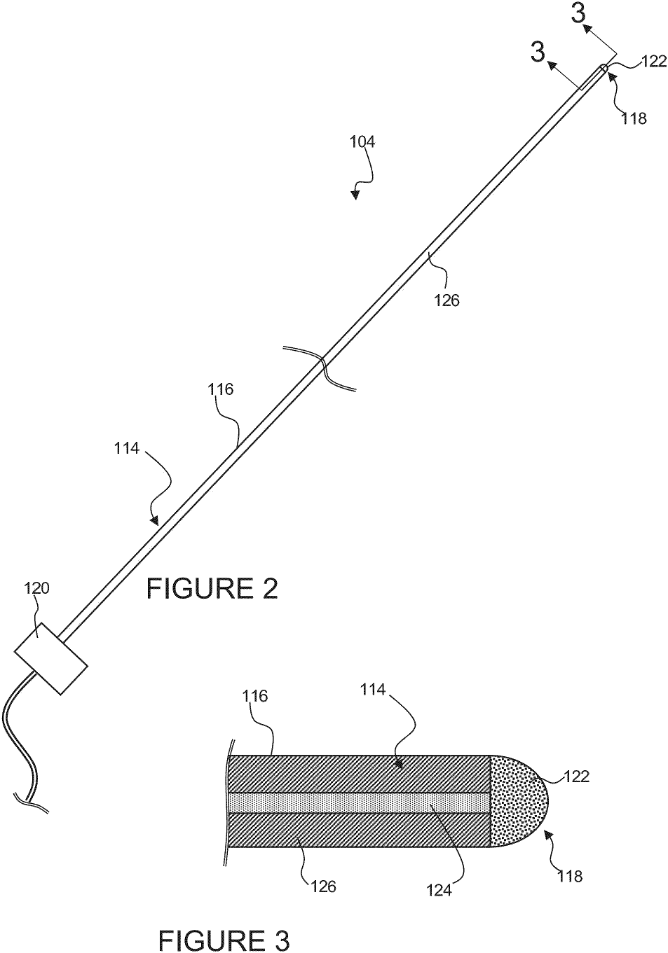

Referring to FIGS. 2 and 3, in the example shown, the RF puncture device 104 includes an elongate member 114 having a shaft 116 and a tip 118, and a hub 120. The tip 118 is at the distal end of the shaft 116, and the hub 120 is at the proximal end of the shaft 116. The tip 118 includes an RF puncture electrode 122 (also referred to herein as an intracorporeal RF puncture electrode) that is positionable adjacent a target site within a patient’s body (e.g. an atrial septum). The shaft 116 includes an electrical conductor 124 and a layer of electrically insulative material 126 on the electrical conductor 124. The electrical conductor 124 is electrically connected to the RF puncture electrode 122 at a distal end thereof, and is further electrically connectable to the RF output port 110 (e.g. via hub 120 and a cable), for delivering RF energy from the RF generator 102 to the RF puncture electrode 122.

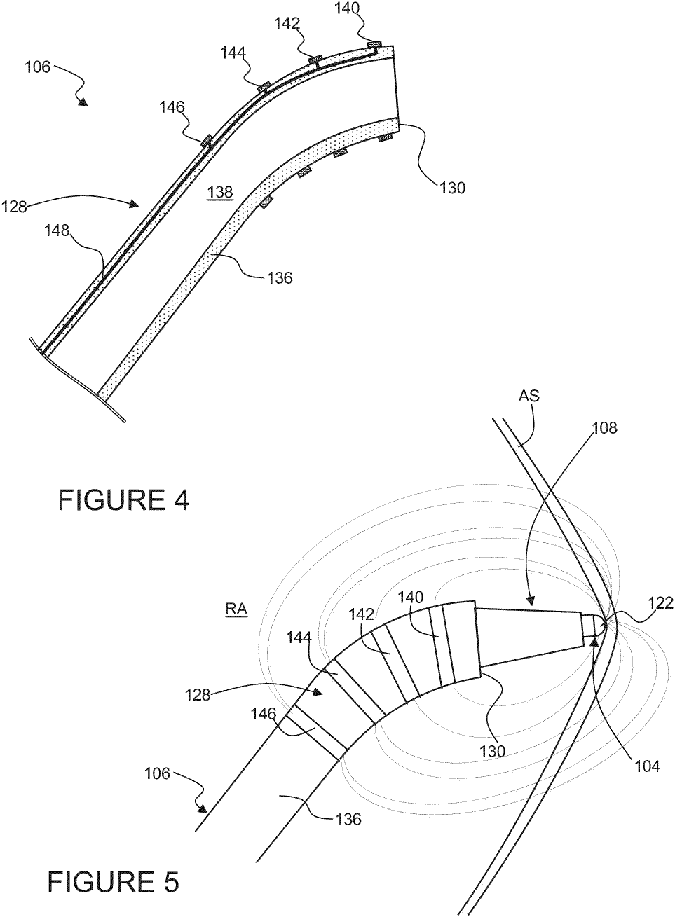

Referring back to FIG. 1, the RF puncture device 104 is advanceable through the sheath 106 and the dilator 108, to position the RF puncture electrode 122 at a target site. The dilator 108 may be any suitable dilator, such as those available from Baylis Medical Company, Inc, and is not described in detail herein. In the example shown, the sheath 106 includes a sheath distal portion 128 that is positionable proximate the target site and that defines a sheath distal end 130, a sheath proximal portion 132 that is opposite the sheath distal portion 128 and that defines a sheath proximal end 134, a sheath sidewall 136 extending between the sheath distal end 130 and the sheath proximal end 134, and a sheath lumen 138 (shown in FIG. 4) defined by the sheath sidewall 136 and extending between the sheath distal end 130 and the sheath proximal end 134.

Referring to FIG. 4, in the example shown, the sheath 106 includes a set of IG electrodes (i.e. a first IG electrode 140, a second IG electrode 142, a third IG electrode 144, and a fourth IG electrode 146). The IG electrodes 140-146 are positionable within the patient’s body proximate the target site, and are electrically connectable to the ground return port 112 of the RF generator 102 (not shown in FIG. 4) for returning current to the RF generator 102. In the example shown, the IG electrodes 140-146 are in the form of ring electrodes that are fixed to the sheath sidewall 136 (e.g. by adhesives and/or friction) in the sheath distal portion 128, and extend circumferentially around the sheath 136. The sheath 106 further includes a ground return wire 148 for electrically connecting the IG electrodes 140-146 to the ground return port 112. For simplicity, only a single ground return wire 148 is shown/ however, the IG electrodes may each have a separate ground return wire associated therewith.

In order to minimize or reduce heating of the IG electrodes 140-146 in use, the IG electrodes 140-146 may have a relatively large surface area (i.e. a surface area that is greater than the surface area of the RF puncture electrode 122).

The IG 140-146 electrodes may, for example, be fabricated from a platinum iridium alloy.

In the example shown, the sheath 106 includes four IG grounding electrodes. In alternative examples, another number of IG grounding electrodes may be used, such as a single IG grounding electrode. However, the use of multiple IG electrodes may reduce the risk of lesion formation if the IG electrode is in contact with tissue.

In the example shown, the IG grounding electrodes 140-146 are ring electrodes that extend around the circumference of the sheath 106. In alternative examples, the IG electrodes may be another shape. For example, the IG electrodes may be positioned on only the concave side of the sheath 106, in order to reduce the risk of contacting tissue.

In further examples, in order to reduce or minimize the risk of the IG electrodes 140-146 contacting tissue, a protective cage may be provided around the IG electrodes 140-146.

In further examples, the RF generator 102 may be programmed to reduce the risk of tissue damage due to the IG electrodes 140-146 touching non-target tissue. For example, the RF generator 102 may be configured to determine the impedance of the grounding circuit, and deactivate if the impedance indicates that one or more of the IG electrodes 140-146 is in contact with tissue.

In an alternative embodiment, electrodes 140-146 of the sheath may be configured to deliver RF energy, while electrode 122 of the puncture device may be configured as a ground.

Referring now to FIG. 5, in use, the RF puncture device 104, sheath 106, and dilator 108 can be advanced towards a target site within a patient’s body. In the example shown, the target site is an atrial septum (AS), and the RF puncture device 104, sheath 106, and dilator 108 can be advanced towards the atrial septum via the femoral vein (not shown), optionally by first advancing the sheath 106 and then advancing the dilator 108 and RF puncture device 104 through the sheath 106. As shown in FIG. 5, the RF puncture electrode 122 of the RF puncture device 104 can be positioned in contact with the target site. Further, the IG electrodes 140-146 can be positioned proximate and spaced from the target site. In the example shown, the IG electrodes 140-146 are positioned within the right atrium (RA), proximally of the RF puncture electrode 104. The RF generator 102 (not shown in FIG. 5) can then be activated, and RF energy can be delivered from the RF outlet port 110 of the RF generator 102 to the RF puncture electrode 122, to puncture the target site. Current can then be returned to the IG electrodes 140-146, and delivered from the IG electrodes 140-146 to the ground return port 112 of the RF generator 102. The return current is shown schematically in dotted line in FIG. 5. When the RF puncture electrode 122 has passed through the atrial septum, the delivery of RF energy can be ceased, and the dilator 108 can then be advanced through the puncture to dilate the puncture. The sheath 106 can then be advanced through the puncture, and the remainder of the medical procedure can be carried out.

Notably, in the example shown, the RF puncture electrode 122 and the IG electrodes 140-146 are positioned on the same side of the target site, and in the same body cavity (i.e. the right atrium).

In an alternative embodiment where the proximally positioned electrodes 140-146 are configured to deliver RF energy, while distally positioned electrode 122 would create a reversed direction of current, opposite of that described in FIG. 5. In other words, the direction of current would move forward (proximal to distal).

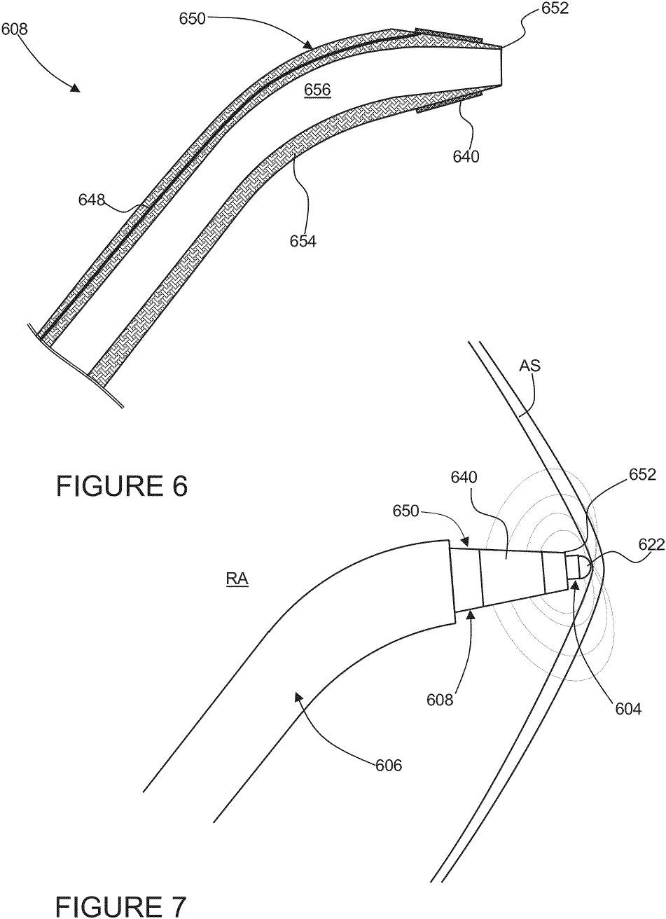

Referring now to FIGS. 6 and 7, an alternative system is shown, in which the IG electrodes are incorporated into a dilator 608. In FIG. 6, features that are like those of FIGS. 1 to 5 will be referenced with like reference numerals, incremented by 500.

Referring to FIG. 6, the dilator 608 includes dilator distal portion 650 that tapers in diameter towards a dilator distal end 652, a dilator proximal portion (not shown) that is opposite the dilator distal portion 650 and that defines a dilator proximal end (not shown). A dilator sidewall 654 extends between the dilator distal end 652 and the dilator proximal end. A dilator lumen 656 is defined by the dilator sidewall 654 and extends between the dilator distal end 652 and the dilator proximal end. An IG electrode 640 is fixed to the dilator sidewall 654 in the dilator distal portion 650 (e.g. using an adhesive, friction, or embedding), and a ground return wire 648 is provided for electrically connecting the IG electrode 640 to the ground return port of the RF generator (not shown).

In the example of FIG. 6, the IG electrode 640 is a ring electrode that extends circumferentially around the dilator sidewall 654. In alternative examples, the IG electrode may be another shape and may be otherwise positioned. For example, the dilator may include a structural hypotube, and an electrically exposed portion of the hypotube may form the IG electrode. Furthermore, in the example of FIG. 6, the dilator 608 includes only a single IG electrode 640. In alternative examples, the dilator 608 may include another number of IG electrodes.

Referring to FIG. 7, in use, the RF puncture device 604, sheath 606, and dilator 608 can be advanced towards a target site within a patient’s body. In the example shown, the target site is an atrial septum (AS), and the RF puncture device 604, sheath 606, and dilator 608 can be advanced towards the atrial septum via the femoral vein (not shown), optionally by first advancing the sheath 606 and then advancing the dilator 608 and RF puncture device 604 through the sheath 606. As shown in FIG. 7, the RF puncture electrode 622 of the RF puncture device 604 can be positioned in contact with the target site. Further, the IG electrode 640 can be positioned proximate and spaced from the target site. In the example shown, the IG electrode 640 is positioned within the right atrium (RA), proximally of the RF puncture electrode 622. The RF generator (not shown in FIG. 7) can then be activated, and RF energy can be delivered from the RF outlet port of the RF generator to the RF puncture electrode 622, to puncture the target site. Current can then be returned to the IG electrode 640, and delivered from the IG electrode 640 to the ground return port of the RF generator. The return current is shown schematically in dotted line in FIG. 7. When the RF puncture electrode 622 has passed through the atrial septum, the delivery of RF energy can be ceased, and the dilator 608 can then be advanced through the puncture to dilate the puncture. The sheath 606 can then be advanced through the puncture.

In an alternative embodiment, the dilator electrode 640 is configured to deliver RF energy while electrode 622 is configured as a ground electrode. In this embodiment, the direction of current is reversed compared to that described in FIG. 7. In other words, the current moves proximal to distal, in a forward direction.

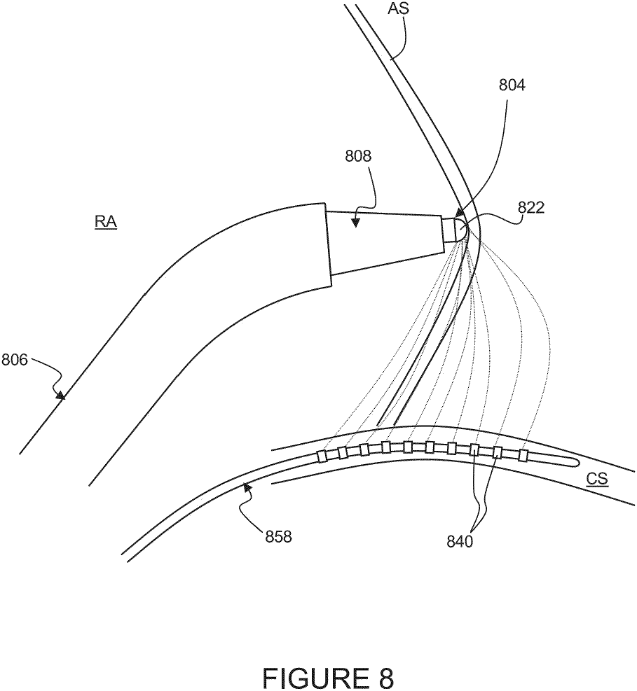

Referring now to FIG. 8, an alternative system is shown, in which IG electrodes are incorporated into a diagnostic catheter 858. In FIG. 8, features that are like those of FIGS. 1 to 5 will be referenced with like reference numerals, incremented by 700.

As shown in FIG. 8, a diagnostic catheter 858 is provided that includes a set of IG electrodes 840 (only two of which are labelled), which are electrically connected to the ground return port of the RF generator (not shown). In use, the diagnostic catheter 858 can be positioned, for example, in the coronary sinus (CS) of the heart, with the IG electrodes 840 in proximity to the atrial septum (AS). The RF puncture device 804, sheath 806, and dilator 808 can be advanced towards a target site within a patient’s body, which in the example shown is the atrial septum. The RF puncture device 804, sheath 806, and dilator 808 can be advanced towards the atrial septum via the femoral vein (not shown), optionally by first advancing the sheath 806 and then advancing the dilator 808 and RF puncture device 804 through the sheath 806. The RF puncture electrode 822 of the RF puncture device 804 can be positioned in contact with the target site. The RF generator (not shown in FIG. 8) can then be activated, and RF energy can be delivered from the RF outlet port of the RF generator to the RF puncture electrode 822, to puncture the target site. Current can then be returned to the IG electrodes 840, and delivered from the IG electrodes 840 to the ground return port of the RF generator. The return current is shown schematically in dotted line in FIG. 8. When the RF puncture electrode 822 has passed through the atrium, the delivery of RF energy can be ceased, and the dilator 808 can then be advanced through the puncture to dilate the puncture. The sheath 806 can then be advanced through the puncture.

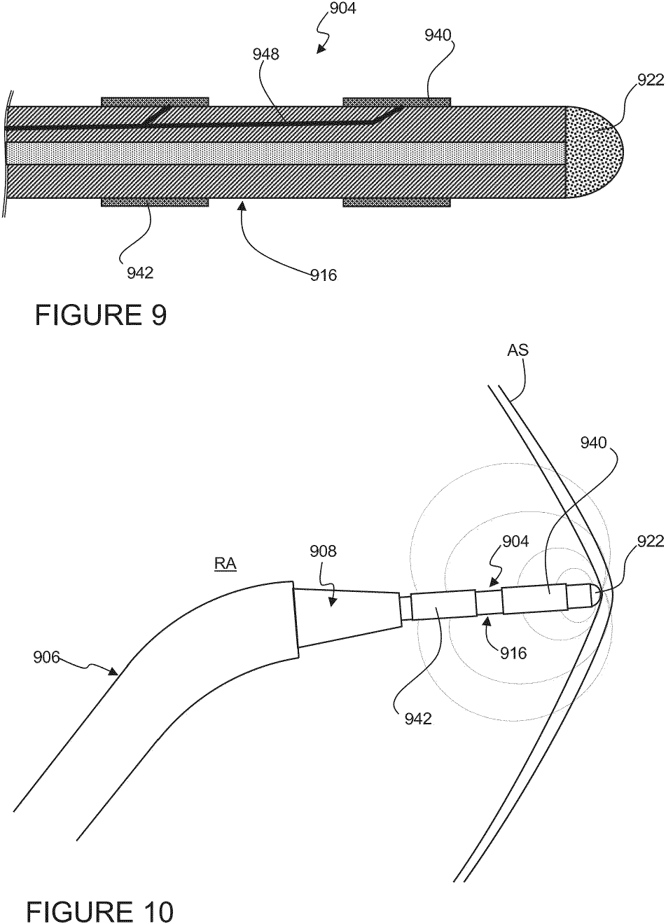

Referring now to FIGS. 9 and 10, an alternative system is shown, in which IG electrodes are incorporated into the RF puncture device 904 itself. In FIGS. 8 and 9, features that are like those of FIGS. 1 to 5 will be referenced with like reference numerals, incremented by 800.

Referring first to FIG. 9, in the example shown, the RF puncture device 904 includes a first IG electrode 940 and a second IG electrode 942, which are in the form of ring electrodes that are received on the shaft 916 and spaced proximally from the RF puncture electrode 922. The shaft 916 further includes a ground return wire 948 that is electrically connected to the IG electrodes 940, 942 and is electrically connectable to the ground return port for returning the current to the RF generator (not shown in FIGS. 9 and 10).

Referring to FIG. 10, the RF puncture device 904, sheath 906, and dilator 908 can be advanced towards a target site within a patient’s body. In the example shown, the target site is an atrial septum (AS), and the RF puncture device 904, sheath 906, and dilator 908 can be advanced towards the atrial septum via the femoral vein (not shown), optionally by first advancing the sheath 906 and then advancing the dilator 908 and RF puncture device 904 through the sheath 906. Alternatively, the RF puncture device 904, sheath 906, and dilator 908 may be advanced toward the atrial septum via a superior or other approach. As shown in FIG. 9, the RF puncture electrode 922 of the RF puncture device 904 can be positioned in contact with the target site. Further, the IG electrodes 940, 942 can be exposed by advancing the IG electrodes 940, 942 proud of the dilator 908 and sheath 906. The RF generator (not shown in FIG. 10) can then be activated, and RF energy can be delivered from the RF outlet port of the RF generator to the RF puncture electrode 922, to puncture the target site. Current can then be returned to the IG electrodes 940, 942, and delivered from the IG electrodes 940, 942 to the ground return port of the RF generator. The return current is shown schematically in dotted line in FIG. 10. When the RF puncture electrode 922 has passed through the atrial septum, the delivery of RF energy can be ceased, and the dilator 908 can then be advanced through the puncture to dilate the puncture. The sheath 906 can then be advanced through the puncture.

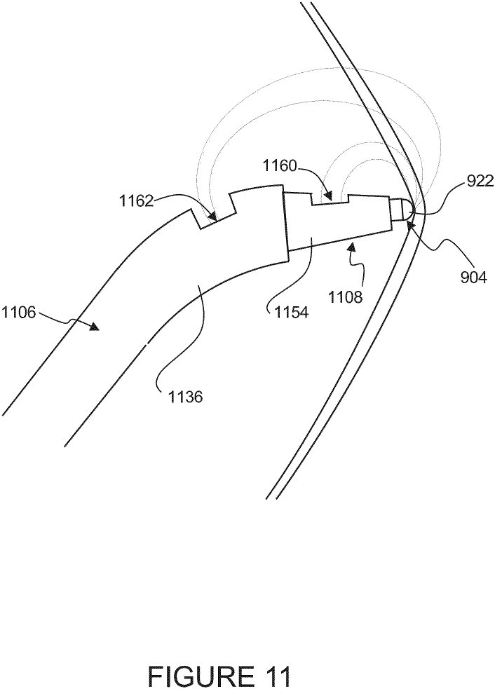

Referring now to FIG. 11, an example is shown in which the RF puncture device 904 of FIG. 9 is used with an alternative dilator 1108 and alternative sheath 1106. The dilator 1108 includes a first window 1160 and a second window (not visible), which extend radially through the dilator sidewall 1154 from an outer surface of the dilator through to the lumen 1156. The sheath 1106 includes a third window 1162, which extends radially through the sheath sidewall 1136 from an outer surface of the sheath 1106 through to the lumen of the sheath 1106. When the dilator 1108 is inserted into the sheath 1106 to the position shown in FIG. 11, the second widow is aligned with the third window 1162. Furthermore, when the RF puncture device 904 is inserted into the dilator 1108 to the position shown in FIG. 11, the first IG electrode 940 (not visible in FIG. 11) is aligned with the first window 1160, and the second IG electrode 942 (not visible in FIG. 11) is aligned with the second window and third window 1162, so that the IG electrodes 940, 942 are electrically exposed.

In an alternative embodiment, the proximally located electrodes 940, 942 640 is configured to deliver RF energy while electrode 922 is configured as a ground electrode. In this embodiment, the direction of current is reversed compared to that described in FIG. 9 to FIG. 11. In other words, the current moves proximal to distal, in a forward direction.

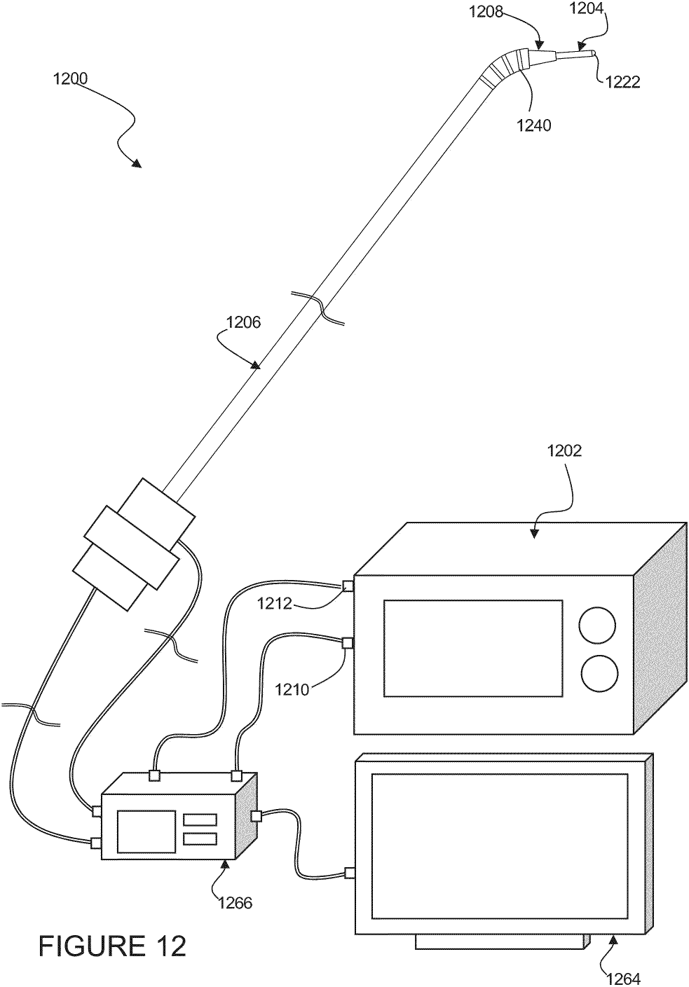

Referring now to FIG. 12, an alternative example of a system is shown. In FIG. 12, features that are like those of FIGS. 1 to 5 will be referenced with like reference numerals, incremented by 1100.

The system 1200 is similar to that of FIG. 1, and includes an RF generator 1202, an RF puncture device 1204 including an RF puncture electrode 1222, a sheath 1206 including a set of IG electrodes 1240 (only one of which is labelled), and a dilator 1208. However, the system further includes an electroanatomical mapping (EAM) system 1264 and a switching device 1266. In some embodiments, the switching device 1266 is integrated into the generator 1202. The IG electrodes 1240 are electrically connectable to the EAM system 1264, for secondary use of the IG electrodes 1240 as EAM electrodes. Particularly, the RF puncture electrode 1222 is electrically connectable to the RF output port 1210 via the switching device 1266. Further the IG electrodes 1240 are electrically connectable to both the EAM system 1264 and the ground return port 1212 via the switching device 1266; however, the switching device 1266 is configured to allow the IG electrodes 1240 to be electrically connected to only one of the ground return port 1212 and the EAM system 1264 at a given time, to allow for the system 1200 to be used either in an EAM mode or a puncture mode. Separate wires may be provided in the sheath 1206 for connecting the IG electrodes to the EAM system 1264 and the ground return port 1212. In an alternative embodiment, the RF puncture electrode 1222 is electrically connectable to both the EAM system 1264 and the RF output port 1210 via the switching device 1266.

The switching device 1266 can optionally further be configured to allow a secondary device (e.g. a grounding pad) be used as a grounding electrode, rather than the IG electrode 1240.

The switching device 1266 and EAM system 1264 of FIG. 12 can additionally or alternatively be used with the systems shown in FIGS. 1 to 11.

In any of the above examples, an EEPROM (electrically erasable programmable read-only memory) may be incorporated into the device that includes the IG electrode (e.g. the dilator, the sheath, the RF puncture device, or the diagnostic catheter).

In any of the above embodiments, a separate device may be used with the puncturing assembly (that is, the puncturing device, and/or dilator, and/or sheath) which comprises a return electrode throughout the procedure. For example, a separate catheter comprising an electrode (or multiple electrodes) configured to act as a ground may be positioned within the body during the procedure.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A system for tissue puncture, comprising:
a radiofrequency (RF) generator comprising an RF output port and a ground return port;
an RF puncture device comprising an elongate member having a shaft and a tip, wherein the tip comprises an intracorporeal RF puncture electrode that is positionable adjacent a target site within a patient’s body, and the shaft comprises a first electrical conductor that is electrically connected to the intracorporeal RF puncture electrode and is electrically connectable to the RF output port for delivering RF energy from the RF generator to the intracorporeal RF electrode; and
at least a first intracorporeal grounding electrode that is positionable within the patient’s body proximate the target site, wherein the first intracorporeal grounding electrode is electrically connectable to the ground return port for returning current to the RF generator.

2. The system of claim 1, further comprising an intracorporeal accessory comprising the first intracorporeal grounding electrode.

3. The system of claim 2, wherein:
the intracorporeal accessory comprises a sheath though which the RF puncture device is advanceable to position the RF puncture electrode at the target site, wherein the sheath comprises sheath distal portion that is positionable proximate the target site and that defines a sheath distal end, a sheath proximal portion that is opposite the sheath distal portion and that defines a sheath proximal end, a sheath sidewall extending between the sheath distal end and the sheath proximal end, and a sheath lumen defined by the sheath sidewall and extending between the sheath distal end and the sheath proximal end; and
the first intracorporeal grounding electrode is fixed to the sheath sidewall in the sheath distal portion, and the sheath further comprises a ground return wire for electrically connecting the first intracorporeal grounding electrode to the ground return port.

4. The system of claim 2, wherein:
the intracorporeal accessory comprises a dilator though which the RF puncture device is advanceable to position the RF puncture electrode at the target site, wherein the dilator comprises dilator distal portion that tapers in diameter towards a dilator distal end, a dilator proximal portion that is opposite the dilator distal portion and that defines a dilator proximal end, a dilator sidewall extending between the dilator distal end and the dilator proximal end, and a dilator lumen defined by the dilator sidewall and extending between the dilator distal end and the dilator proximal end; and
the first intracorporeal grounding electrode is fixed to the dilator sidewall in the dilator distal portion, and the dilator further comprises a ground return wire for electrically connecting the first intracorporeal grounding electrode to the ground return port.

5. The system of claim 2, wherein
the intracorporeal accessory comprises a diagnostic catheter having a catheter distal portion that is positionable proximate the target site and that defines a catheter distal end, a catheter proximal portion that is opposite the catheter distal portion and that defines a catheter proximal end, and a catheter sidewall extending between the catheter distal end and the catheter proximal end;
the first intracorporeal grounding electrode is fixed to the catheter sidewall in the catheter distal portion, and the catheter further comprises a ground return wire for electrically connecting the first intracorporeal grounding electrode to the ground return port.

6. The system of claim 1, wherein the RF puncture device comprises the first intracorporeal grounding electrode.

7. The system of claim 6, wherein the shaft comprises a ground return wire that is electrically connected to the intracorporeal grounding electrode and is electrically connectable to the ground return port for returning the current to the RF generator.

8. The system of claim 6, wherein
the system further comprises a dilator though which the RF puncture device is advanceable to position the RF puncture electrode at the target site, wherein the dilator comprises dilator distal portion that tapers in diameter towards a dilator distal end, a dilator proximal portion that is opposite the dilator distal portion and that defines a dilator proximal end, a dilator sidewall extending between the dilator distal end and the dilator proximal end, and a dilator lumen defined by the dilator sidewall and extending between the dilator distal end and the dilator proximal end;
in the dilator distal portion, the dilator sidewall comprises a first window extending radially therethrough from an outer surface of the dilator to the dilator lumen; and
when the RF puncture device is advanced through the dilator to position the RF puncture electrode at the target site, the first intracorporeal grounding electrode is aligned with the first window.

9. The system of claim 8, wherein
the system further comprises a sheath having a sheath distal portion that is positionable proximate the target site and that defines a sheath distal end, a sheath proximal portion that is opposite the sheath distal portion and that defines a sheath proximal end, a sheath sidewall extending between the sheath distal end and the sheath proximal end, and a sheath lumen defined by the sheath sidewall and extending between the sheath distal end and the sheath proximal end;
the RF puncture device and the dilator are advanceable through the sheath lumen to position the dilator distal portion proud of the sheath distal end and to position the RF puncture electrode proud of the dilator distal end and the sheath distal end and at the target site;
in the sheath distal portion, the sheath sidewall comprises a second window extending radially therethrough from an outer surface of the sheath to the sheath lumen; and
when the dilator is advanced through the sheath and the RF puncture device is advanced through the dilator to position the RF puncture electrode at the target site, the second intracorporeal grounding electrode is aligned with the second window.

10. The system of claim 1 further comprising an electroanatomical mapping (EAM) system to which the first intracorporeal grounding electrode is electrically connectable for use of the first intracorporeal grounding electrode as an EAM electrode.

11. The system of claim 10, further comprising a switching device, wherein
the RF puncture electrode is electrically connectable to the RF output port via the switching device;
the first intracorporeal grounding electrode is electrically connectable to the EAM system via the switching device; and
the first intracorporeal grounding electrode is electrically connectable to the ground return port via the switching device.

12. The system of claim 10, wherein the switching device is configured to allow the first intracorporeal grounding electrode to be electrically connected to only one of the ground return port and the EAM system at a given time.

13. A method for tissue puncture comprising:
a. advancing a radiofrequency (RF) puncture device towards a target site within a patient’s body and positioning an intracorporeal RF puncture electrode of the RF puncture device in contact with the target site;
b. advancing a first intracorporeal grounding electrode into the patient’s body and positioning the first intracorporeal grounding electrode proximate and spaced from the target site;
c. delivering RF energy from an RF outlet port of an RF generator to the RF puncture electrode, to puncture the target site; and
d. and returning current to the first intracorporeal grounding electrode and delivering the current from the first intracorporeal grounding electrode to a ground return port of the RF generator.

14. The method of claim 13, wherein in step a., the intracorporeal RF puncture electrode is positioned in a body cavity, and in step b., the first intracorporeal grounding electrode is positioned in the body cavity.

15. The method of claim 13, wherein
step b. comprises advancing a sheath into the patient’s body, wherein a distal portion of the sheath comprises the first intracorporeal grounding electrode; and
step a. comprises advancing the RF puncture device through the sheath.

16. The method of claim 13, wherein
step b. comprises advancing a dilator into the patient’s body, wherein a distal portion of the dilator comprises the first intracorporeal grounding electrode; and
step a. comprises advancing the RF puncture device through the dilator.

17. The method of claim 13, wherein
step b. comprises advancing a diagnostic catheter into the patient’s body, wherein a distal portion of the diagnostic catheter comprises the first intracorporeal grounding electrode.

18. The method of claim 13, wherein a distal portion of the RF puncture electrode comprises the first intracorporeal grounding electrode, and step a. and step b. are carried out concurrently by advancing the RF puncture device towards the target site.

19. The method of claim 13, further comprising:
before or after steps c. and d., connecting the first intracorporeal grounding electrode to an electroanatomical mapping system and using the first intracorporeal grounding electrode for electroanatomical mapping.

20. The method of claim 13, further comprising:
before or after steps c. and d., connecting the RF puncture electrode to an electroanatomical mapping system and using the RF puncture electrode for electroanatomical mapping.
